(19) United States
(12) Patent Application Publication
(10) Pub. No.: US 2015/0297122 A1
(43) Pub. Date: Oct. 22, 2015

(54) METHOD OF SCREENING TINNITUS BY OCCLUDING A PATIENT'S OUTER EARS AND ANALYZING PATIENT FEEDBACK TO LOCATE TO AFFLICTED AREA(S)

(71) Applicant: Anjan Muhury, Long Beach, CA (US)

(72) Inventor: Anjan Muhury, Long Beach, CA (US)

(21) Appl. No.: 14/258,845

(22) Filed: Apr. 22, 2014

(51) Int. Cl. A61B 5/12

(52) U.S. Cl. CPC A61B 5/128

(57) ABSTRACT

Instructing a patient to listen carefully for specific sounds while his or her ear canals are occluded. Occluding the cannels and observing the patient to determine when he or she recognizes a sound described in the instruction, opening the patient's ear cannels, questioning about the specific sound perceived and recording the results.

### BACKGROUND

1. Field of the Invention

The present invention relates generally to afflictions of the ear and particularly to a protocol for screening a tinnitus patient to determine the results of the pathology.

2. Description of Prior Art

Tinnitus is one of the most elusive conditions facing health care professionals. It is an auditory perception not directly produced externally. Commonly described as by the patient as perceiving a hissing, roaring, ringing or whooshing sound in one or both ears and typically referred to as tinnitus aurum or in the head, called tinnitus crani. Tinnitus can be broadly described as either objective or subjective. Objective tinnitus accounts for less than five percent of the overall tinnitus cases and is often associated with vascular or muscular disorders. Subjective tinnitus is audible only to the patient and is much more common, accounting for ninety five percent of tinnitus cases. Subjective tinnitus is a symptom associated with practically every known ear disorder and is reported to be present in over eighty percent of individuals with sensorineural hearing loss, which is caused by nerve and/or hair cell damage. Because tinnitus, like pain, is subjective, two individuals may demonstrate identical tinnitus loudness and pitch matches yet may be affected in significantly different ways. Many tinnitus sufferers have difficulty sleeping or concentrating, feel depressed or anxious, report additional problems at work or at home that may contribute to the distress caused by tinnitus.

Tinnitus is common, affecting about ten to fifteen percent of the population. Most tolerate it well, the problem only being significant in about one to two percent of the those afflicted. Tinnitus can be perceived in one or both ears or in the head. It is usually described as a ringing noise but in some patients it takes the form of high pitch whining, electric, busing, hissing, humming, tinging, or a whistling sound or as ticking, clicking, roaring, “crickets” or “tree frogs” or “locusts” or the like.

In some cases patients can perceive actual sound coming from his or her ears. This is called objective tinnitus.

Subjective tinnitus can have many possible causes, but most commonly results from otologic disorders, the same conditions that often causes hearing losses.

Screening of patients complaining that they perceive tinnitus-like sounds typically leaves a healthcare professional to speculate as to what might be the cause, thereby, oftentimes leaving a patient untreated. Left untreated the affliction can become so serious and stressful as to lead to strokes and partially or totally limit the patient's ability to function, sometimes even leading to death.

Often times, the health care professional is left only to treat tinnitus with some type of procedure to mask the noise effect and reduce the consequence discomfort of the patient. It is known that the cause and treatment of tinnitus is especially challenging because conventional testing techniques are unable to accurately and reliably detect and quantify tinnitus.

One common detection and characterization method involves exposing the subject to several different sound patterns and asking the subject which pattern is the most qualitatively similar to the sound her or she perceived. While this method may be beneficial for detecting tinnitus in some subjects, it is not effective where the subject is unable to communicate with the administrator or where the subject cannot perceive all of the effects of the subjects own tinnitus conditions. Because this approach relies solely on the subject to explain his or hers symptoms, this approach is also subject to malingering, a condition where a subject claims to have a medical problem, such as tinnitus, when no such problem actually exists. In recognition of the shortcomings of testing procedures, it has been proposed to analyze a tinnitus condition by analyzing the subject's acoustic startle reflex. This approach, involves various equipment for exposing the patient to a primary sound pattern, having a sound acoustic signal selected to be qualitatively similar to the subjects tinnitus. The subject is exposed to a reflex stimulus following exposure to the following primary sound paten to elicit a stimulus response which is measured by an electrical response sensor. An approach of this type is described in U.S. Pat. No. 8,080,772 to Turner. This system requires rather complicated and expensive paraphernalia and relies extensively on the ability of the health care professional to select a sound pattern similar to what that patient describes he or she is experiencing.

A patient suffering from tinnitus recites symptoms which indicate are known to suggest particular comments such as middle ear structural changes known as tympanic abnormalities otosclerosis, tympanosclerosis, ossicular, disarticulation/disruption, calcification of the middle ear cavity, or affliction of the inner ear cavity, such as cochlear damage to loud sounds, viral cochleitis, labrinithitis, or retrocochlear ailments, by damage to the 8^{th} nerve from cochlea on cochlear nucleous, demylentizleyon of the nerve fibers and the white matter areas, neuronal damage in the temporal of frontal lobe of the brain, nerve damage due to whiplash type injury in the brain or the like.

It is known that various degrees of tinnitus is suffered by different patients. Some patients experience soft ringing while sleeping or drowsy, something that is typically masked during daytime activity by environmental sounds. The patient is required to concentrate with great focus to even perceive, identify and explain certain sounds. A ringing tone may be experienced in many different environmental settings situations but patients typically adapt allowing the tone to be tolerated.

When the tone increases to a loud ringing it may cause distraction during conversations and everyday activity and the mental stress and anxiety levels typically merit attention as the patient is not allowed to focus on cognitive actions. Consequently, there is a need to examine the variations in the sound level and types of characteristics of sound perceived by the patient. This is best achieved by isolation in a sound both or a low decibel environment.

From my experiments, a buzzing, humming or ringing sound experienced by a patient when the ear canals are occluded typically suggests structural changes in the middle ear. Buzzing or humming typically indicates central/neurological issues. Ringing or roaring tinnitus suggest Meniere's symptoms, round window rupture/perilymph fistulas (in cases where fullness is present), or aneurisms (vascular) in arteries of the brain. Chirping like cricket sounds or locusts suggest extreme damage to cochlear cells. Hallucinations suggests possible damage in the media-dorsal pre-frontal cortex and may also be accompanied by cognitive deficits like executive functions, episodic memory retrieval.

My protocol is typically administered in a sound isolated area as in a traditional sound booth. Beforehand the health care professional explains the procedure. The explanation is made that the external ear canals will be occluded and the patient is to focus on his or her perception of changes in the sounds perceived. The patient's ears are then occluded as by ear plugs or gloved fingers and, pursuant to instructions, is to nod or otherwise indicate when he or she has perceived the expected sound. The plugs are then removed from the patients ear canals and the patient questioned what he or she perceived. These steps may be repeated twice for better accuracy and to avoid erroneous readings.

My protocol is totally new for utilizing the perceived intensity and characteristic of sound perceived by the patient with the ear canals occluded as being instructive of the area of the ear which is likely afflicted.

In listening to a patient complaining of tinnitus, I have counseled and advised of the details of the protocol. I advised the patient that he or she will isolated in an acoustical room and his or her ear canals occluded. The patient is then to focus on the sounds perceived and suggestions are made of the of what might be expected as the, for instance, humming/buzzing which may reduce or shut off. This suggests that the ideology is in the middle ear and a referral to an otologists may be indicated for otsclerosis-tympanoscleorosis. I suggested to the patient that he or she also listen for a perceived humming/buzzing which persisted at the same level. In that instance my experience suggest that the etiology may be may be retro cochlear (along the eight auditory nerve/elsewhere in the grey matter). I suggest that if the perceived sound is harsh buzzing/loud humming that does not change in intensity over time ideology maybe retro cochlear (white matter disease, like MS). If the complaint is more like roaring/sea shell tinnitus that increases with the plugging effect, the ideology lies in the sclera media of the prolcea, and referral is thus indicated. I also inquire of the patient of vertigo, fullness and the fluctuating hearing. If the roaring is steady and accompanied with headaches but no fullness or vertigo, then neurological referral may be indicated by for aneurism. If the complaint is chirping/cricket type sounds increasing with plugging of the ears, the ideology is confirmed in the cochlea. Where the patient reports hallucinations or inner voices, experiences random events that cannot be verified with the tinnitus screening test but can be studied and judged with cognitive defects notices or reported a referral to a neurologist may be indicated for aneurisms in the pre-frontal cortisies.

### SUMMARY OF THE INVENTION

The present invention includes the steps of instructing a patient on a procedure to be administered involving the characteristics of the noises which might be perceived and then isolating the patient from ambient noise. The patient's ear canals are then occluded for a short time to listen closely and the then patient removed from isolation and the ear canals opened. The patient is then administered a series of questions about the characteristics of noises perceived and those results recorded to indicate what portion or section of the ear may be the cause of the tinnitus problem to thus select a specialist such as an otologist, or a neurologist or other specialists for further treatment.

Through many years of experience and experimentation, I have determined that there are certain levels and intensity of sounds perceived by a tinnitus patient when his or her ears are occluded. The characteristic of which sounds are often times indicative of the element suffered and the treatment was made beneficial to the patient. While these indicators may have been available to those working in the art, I am unaware of any professional who has isolated the indicators and identified with the particular area of the ear which might come in some instances treated for improvement.

### BRIEF DESCRIPTION OF THE DRAWINGS

FIG. 1 is a schematic view of a patient's ear;

FIG. 2 is a logic chart showing the screening test of the present invention; and

FIG. 3 is a further logic diagram depicting steps of analyzing and recording the results of the tests of the present invention.

### DETAILED DESCRIPTION OF THE PREFERRED EMBODIMENTS

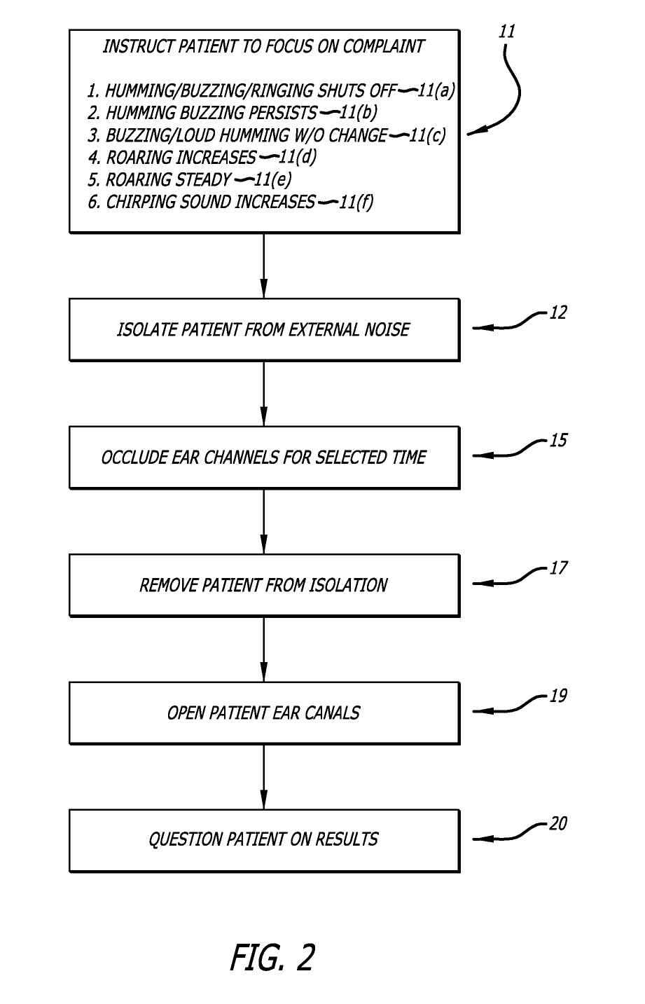

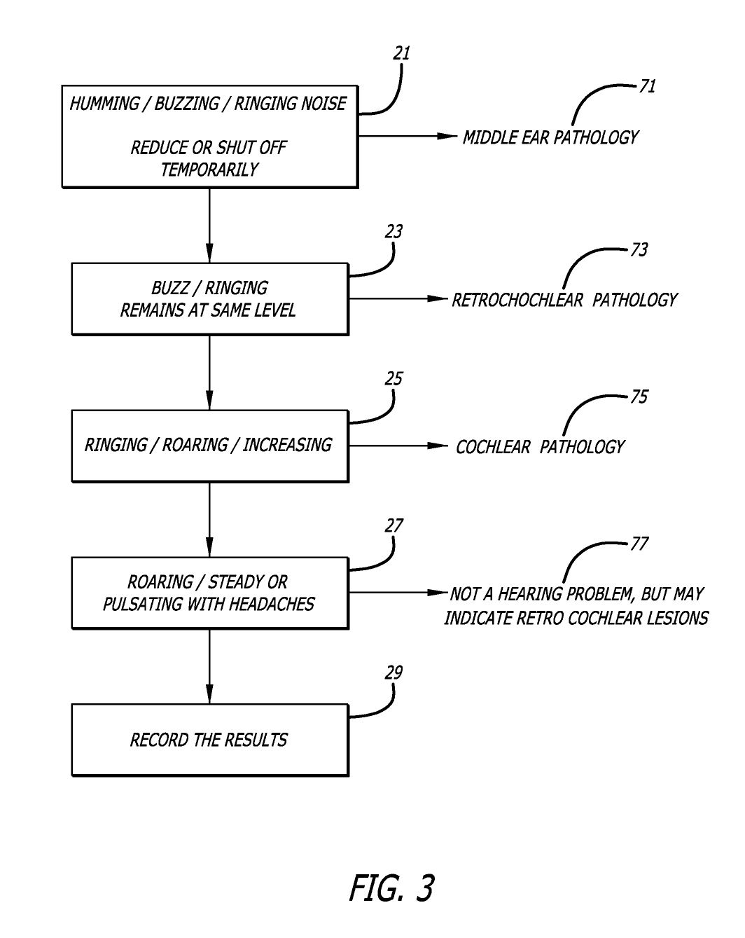

Referring to FIGS. 2 and 3, the present invention involves a health care professional instructing a patient about the procedure at step 11 and then isolating the patient from ambient noise at step 12 where the patients ear canals are occluded at 15. After about fifteen seconds the patient is removed from isolation at 17 and the ear canals opened at 19 and the outcome determined at 20. The patient is administered a series of questions at 21, 23, 25, 27, and 29 and the results recorded at 31 for analysis as to the source of the problem and necessary referral to a further specialist.

The perception of the tinnitus sound range can include buzzing, humming and soft ringing, which suggest middle ear pathologies, ringing which suggest cochlear pathologies or hard buzzing, loud ringing, hallucinations, all of which suggest centralization perception within the skull and not necessarily heard in the left or right ears. My screening test protocol is the first to screen patient's perception in an effort to locate the possible source of the ailment for appropriate medical referral.

From my studies I have discovered that, if the patient perceives tinnitus as increasing the pathology is fixed in the cochlear hair cells. And if the perception of the tinnitus is reported as having been reduced or dropped significantly, the source of ailment is likely in the middle ear as the drop is typically due to stoppage of osicular vibration while the ear canals are occluded. In the event the patient has reported there has been no change in the sound perceived, my research indicates that the etiology is existing retrocochlear or ailment in other brain areas.

The results of my research reveals that where the perception of tinnitus reduces or drops significantly or where there is no change in perception, referral should be made to a otologist or neurologist for other diagnostic tests. Since there is no cure for damage to the cochlear hair cells patients reporting tinnitus increases should be referred for audiology or hearing aide evaluation or counseling for hearing protection.

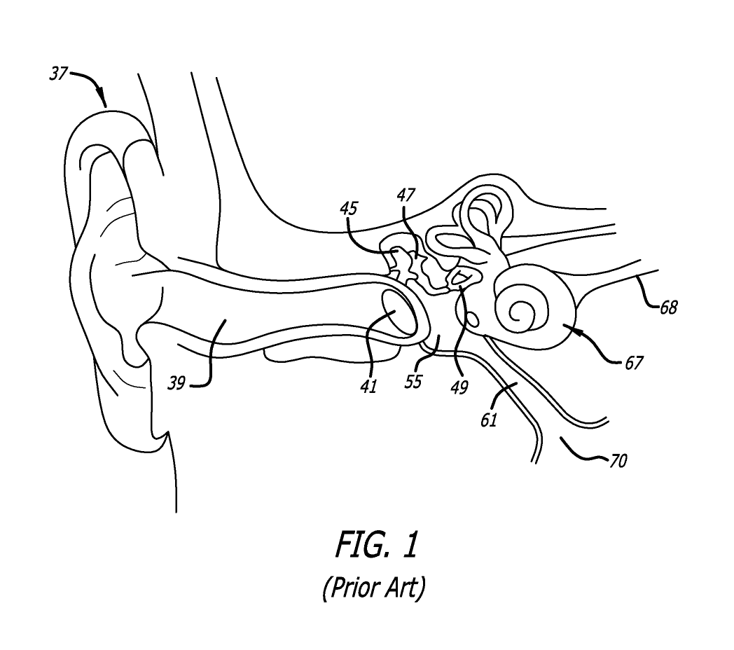

Referring FIG. 1, a human ear tend has a pinia 37 external ear canal (external meatus) 39 tympanic membrane 41 (eardrum). The auditory sounds in the environment enter the external canal 39 and acoustically vibrate the tympanic membrane 41. Ossicles 43, also called ossicular chain, and more particularly the malleous 45, the incus 47 and stapes 49 are connected to the tympanic membrane 16 and transmit the acoustic vibrations representing the auditory sound through the middle ear cavity 53. The middle ear cavity 53 is vented to the nasopharynx by the Eustachian tube 61. The ossilus 43 then transmits the vibration to the inner ear 65, represented by the cochlea 67. The cochlea 67 is a spiral, fluid filled organ which transforms the acoustic vibration then to nerve discharges transmitted to the brain via the 8^{th} auditory nerve bundle 68.

Referring back to FIGS. 2 and 3, the patient is typically counseled and advised of the sounds indicative of tinnitus. In my procedure, I explain to the patient at step 11 that his or her ear cannels would be occluded and encourage the patient to listen carefully and seek to identify the characteristics and continuous of the sounds perceived with the occluded ear.

To improve the effectiveness the patient is removed from ambient noise at step 12, as by isolating in a sound booth or the like and observe through a window to monitor reactions of the patients. In the instructions the patient is alerted to signal when a characteristic one of the sounds is recognized so that the patient may be removed from isolation and the ear cannels open.

I alert the patient to focus carefully and listen for sounds which come with the ear canals occluded, might be indicative of the tinnitus suffered. For instance, at step 11 I advise the patient to listen for a humming/buzzing/ringing sound which may either shut off at step 11(a) over time or persist for a period of time at 11(b). I additionally tell the patient to listen for a bussing or loud humming which is consistent with an intensity without change over a period of time at 11(c) and to be alert for a roaring sound which increases over time 11(d) or a roaring sound which remains steady over a period of time at 11(f). Finally, I alert the patient to listen for a chirping sound which may increase over time 11(g).

I occlude the patient's ear as by ear plugs or gloved fingers for a short period of time at 15 while the patient listens and maybe signals through a window or the like when he or she recognizes a sound of 11 (a)-(g). I then remove the patient from isolation at 17 and open the ear canals and question the patient at 20 and record the answers at 29.

When the patient is removed from isolation, I then merely ask what sound was perceived or to jog the patient's memory and cognizance, ask each of the questions 21-27. If the patients responds positively to any question a check mark recorded in his or her record and if the patient responds negatively, the words “no” or a circle placed in the record at response to that question to provide a record at 29.

I then retain that information in my medical records for counseling and possible reference to another specialist.

If the answer to the question of whether the patient perceived ringing/buzzing or ringing which reduced or shut off at 21 is positive, I will record in the patient's record that the pathology suggests the affliction is likely in the middle ear 71.

If the patient acknowledges he or she perceived a buzzing or ringing sound remaining constant at the same volume level at 23, I then record that the pathology suggests the problem is likely retrochochlear 73.

If the response is that the ringing/roaring increases at 25, I then indicate in the patient's record that the pathology suggests the problem is likely in the media of cochlea 75.

If the response to whether the patient perceived roaring, either steady or pulsating, along with headaches at 27, I then record that in my opinion the pathology suggests that the problem is not likely a hearing problem but may be retrocochlear lesions at 77.

From the information recorded in this protocol a patient may be referred to a selected specialist for treatment.

As will be appreciated by those skilled in the art, the method of the present invention provides for administering a protocol which, is many cases allows for a diagnosis of treatment by a professional which can often lead to relief not heretofore attainable.

From the forgoing it would be appreciated that the method of the present invention provides a reliable and an effective method for screening tinnitus patients to isolate the area likely affected for the purpose of identifying a specialist for further treatment for advising the patient on how best to address the hearing or tinnitus issues.

Although the present invention has been described in detail with regard to the preferred embodiments and drawings thereof, it should be apparent to those of ordinary skill in the art that various adaptations and modifications of the present invention may be accomplished without departing from the spirit and the scope of the invention. Accordingly, it is to be understood that the detailed description and the accompanying drawings as set forth hereinabove are not intended to limit the breadth of the present invention.

I claim:

1. A method of screening a patient for tinnitus including:
a. Instructing the patient that his or her ears will be occluded and to listen carefully for perceived sounds giving examples of humming, buzzing or ringing, which might, after a short period decrease or shut off temporarily the buzzing or ringing at a continuous volume, roaring which increases or holds steady or that increases in volume;
b. isolating the patient from ambient noise;
c. occluding the patents ear channels for a predetermined time;
d. removing the patient from isolation;
e. opening the patient's ear channels;
f. questioning the patent on whether he or she perceived sounds of one of the following nature;
1.) a humming, buzzing or ringing and whether the sound is perceived as reduced in perceived volume or ceased;
2.) a buzz or ringing of constant volume;
3.) ringing or roaring increasing in perceived volume;)
4.) roaring remaining constant or pulsating volume;
g. for any answers to questions f. 1.)-4.), recording the results as the middle ear, retrochochlear, cochlea or not a hearing problem, respectively.

2. The method of claim 1 wherein:
that includes isolating the patient from ambient noises during the time the ear canals are occluded.

3. The method of claim 1 that includes:
instructing the patient on what to expect in the way of sounds and intensities thereof; and
questioning the patient on the sounds perceived relating to the categories of a humming or buzzing sound become a harsh buzz, a roaring sound and a chirping sound.

4. The method of claim 1 wherein:
in the event the patient responds to f. 1.) that the perceived sounds are reduced in intensity;
in step g) recording that the pathology suggests the middle ear.

5. The method of claim 1 wherein:
in the event the patient responds to f. 2.) that he or she perceives the sound as a constant intensity of buzz, recording in step g. retrochochlear.

6. The method of claim 1 wherein:
in the event the patient responds positively to inquiry f. 3.), in step g. recording cochlea.

7. The method of claim 1 wherein:
in the event the patient responds positively to inquiry f. 4.), inquiring if the patient experiences headaches.

8. The method of claim 5 wherein:
If the patient responds that he or she experiences headaches, in step g recording an indication that the pathology suggests the problem is not tinnitus.

9. A method of screening a patient for tinnitus including:
a) instructing the patient that his or her ears will be occluded and to listen carefully for perceived sounds;
b) isolating the patient from ambient noise;
c) occluding the patents ear channels for a predetermined time;
d) removing the patient from isolation;
e) opening the patient's ear channels;
f) questioning the patent on what he or she perceived was heard to solicit answers;
g) recording the patients answers; and
h) from the answer, recommending a treatment.

10. The method of claim 9 wherein:
step a) includes instructing the patient to listen for buzzing or ringing and note if it shuts off.

11. The method of claim 9 wherein:
step a) includes instruction the patient to listen for buzzing without change.

12. The method of claim 9 wherein:
step a) includes listening for roaring and note whether the intensity increases over time.

13. The method of claim 9 wherein:
step a) includes instructing the patient to listen for a roaring sound and note whether it remains steady over time.

14. The method of claim 9 wherein:
step a) includes instructing the patient to listen for a chirping sound.

15. A method for screening a patient for tinnitus including:
a) instructing a patient that his or her ears will be occluded and to listen carefully for perceived sounds giving examples of humming, buzzing, roaring, ringing and to listen for changes in volume over time;
b) occluding the patient's ear canals for a predetermined time;
c) opening the patient's ear canals;
d) questioning the patient on which sounds were perceived and recording the results.
